(19) United States
(12) Patent Application Publication
(10) Pub. No.: US 2007/0128636 A1
(43) Pub. Date: Jun. 7, 2007

(54) Predictors Of Patient Response To Treatment With EGFR Inhibitors

(76) Inventors: Joffre B. Baker, Montara, CA (US); Michael C. Kiefer, Clayton, CA (US)

Correspondence Address: HELLER EHRMAN LLP, 275 MIDDLEFIELD ROAD, MENLO PARK, CA, 94025-3506, US

(21) Appl. No.: 11/566,665

(22) Filed: Dec. 4, 2006

Related U.S. Application Data
(60) Provisional application No. 60/742,702, filed on Dec. 5, 2005.

(51) Int. Cl. C12Q 1/68; G06F 19/00

(52) U.S. Cl. 435/6; 702/20

(57) ABSTRACT

The invention concerns genes and gene sets and methods useful in the prediction of the response of a cancer patient to treatment with an epidermal growth factor receptor (EGFR) inhibitor.

### CROSS-REFERENCE TO RELATED APPLICATIONS

This is a non-provisional application filed under 37 CFR 1.53(b), claiming priority under USC Section 119(e) to provisional Application Ser. No. 60/742,702, filed Dec. 5, 2005 which is incorporated by reference herein.

### BACKGROUND OF THE INVENTION

1. Field of the Invention

The present invention concerns genes and gene sets and methods useful in the prediction of the response of a cancer patient to treatment with an epidermal growth factor receptor (EGFR) inhibitor.

2. Description of Related Art

Until recently, cancer was poorly understood at a molecular level and was generally viewed as a homogenous disease characterized by rapidly proliferating cells. Drugs developed based on this insufficient understanding of cancer biology attacked rapidly dividing cells indiscriminately and, as a result, often exhibited a high degree of toxicity. In most instances, little was known regarding the mechanisms of action of these cytotoxic drugs.

We know now that cancers of individual tissues, which were once regarded as homogenous diseases, result from a spectrum of underlying biological defects. As the detailed biology of cancer has become better understood, a new generation of drugs that target specific aspects of this biology is being developed. Because of their biological specificity, these drugs demonstrate reduced (though often significant) toxicity, but tend to be expensive and are effective in only a subset of patients. Tests that can identify those patients who are likely to respond to particular therapeutic compounds are needed in order to optimize application of targeted drugs and to avoid unnecessary expense and toxic exposure for those patients who are unlikely to respond.

For this reason, there is an emerging trend to develop and commercialize targeted drugs in concert with companion diagnostic tests capable of identifying responsive patients. Trastuzumab (HERCEPTIN®), a monoclonal antibody that recognizes the ERBB2 growth factor receptor is an early example of such a drug. The gene encoding ERBB2, a member of the EGFR family, is amplified in a subset of breast cancers and the resulting overexpression of the receptor contributes to breast cancer etiology. This amplification can be detected at the DNA level using fluorescent in situ hybridization (FISH), or resulting protein overexpression can be detected using immunohistochemistry. Trastuzumab is approved only for patients whose tumors overexpress ERBB2 as measured by one of these tests.

The pharmaceutical industry has recently expended significant effort in the development of drugs targeted to the receptor for EGF, an important positive regulator of cell growth and differentiation. Three drugs that inhibit EGFR have been approved by the United States Food and Drug Administration (FDA) for the treatment of various forms of cancer, and a number of others are in various stages of clinical testing. Gefitinib (IRESSA®, AstraZeneca) and Erlotinib (TARCEVA®, Genentech, Inc.) are FDA approved small molecule tyrosine kinase inhibitors (TKI) that inhibit signaling through the tyrosine kinase domain of EGFR; Cetuximab (ERBITUX®, ImClone Systems, Inc.) is a monoclonal antibody that interferes with EGFR receptor phosphorylation (Sunada, H. et al. Proc Natl. Acad. Sci. U.S.A. 83:3825-9 (1986)). Each of these EGFR inhibitors exhibit efficacy in only a subset of patients who receive them (M. G. Kris et al. J. Am. Med. Assoc. 290:2149-58 (2003); M. Fukuoka J. Clin. Oncol. 21:2237-46 (2003); M. Moroni et al. Lancet Oncol. 6: 279-86 (2005)).

Genetic markers of patient response to TKI have been reported, but these markers have not been proven to predict overall survival (T. J. Lynch et al. N Engl. J. Med. 350:2129-39 (2004); F. Cappuzzo et al. J. Natl. Cancer Inst. 97:643-55 (2005); D. W.Bell et al. J. Clin. Oncol. 23:8081-92 (2005)). Expression markers of patient response to EGFR inhibitors are disclosed in United States Patent Application Publication Nos. 20040157255, published Aug. 12, 2004,; and 20050019785, published Jan. 27, 2005.

Despite earlier advances in the identification of patients who are more likely or less likely to respond to treatment with EGFR inhibitor drugs, additional molecular markers of patient response to EGFR inhibitors are needed. A need exists for a test that can more reliably predict clinical benefit in response to EGFR inhibitors. Without such a test, some patients who might benefit from treatment may not receive the drug, and other patients who are unlikely to benefit may be unnecessarily exposed to toxic side effects, incur unnecessary expense, and/or may experience a delay in being treated with alternative drugs that might prove more effective.

Tests for predicting patient responsiveness to EGFR inhibitors may be configured for one or both of two related purposes. One purpose is to predict the likelihood of response to one particular compound that is an EGFR inhibitor. A gene marker useful in making such a prediction (drug responsiveness marker) may or may not be useful in predicting the likelihood of response to a different EGFR inhibitor. Another purpose would be to predict the likelihood of response to any member of the class of EGFR inhibitors. A test can be based on markers each of which is useful for predicting the responsiveness generally to EGFR inhibitors (class responsiveness markers). Tests can be configured comprising both class responsiveness markers and drug responsiveness markers for one or more specific drug compounds.

### SUMMARY OF THE INVENTION

In one aspect, the present invention concerns a method for predicting the response of a subject diagnosed with EGFR positive cancer to treatment with an EGFR inhibitor, comprising determining the expression level of one or more RNA transcripts or their expression products in a biological sample containing cancer cells obtained from said subject, wherein the RNA transcript is of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in antibody-dependent cell-mediated cytotoxicity (ADCC) and gene markers of immune or inflammatory cells; (vi) genes associated with tumor cell invasion; and (vii) genes characteristic of late stage tumors, wherein
(a) for every unit of increased expression of one or more genes selected from groups (i), (ii), (iii), (iv), and (v), or the corresponding expression product, the subject is predicted to have an increased likelihood of response to treatment with the EGFR inhibitor; and
(b) for every unit of increased expression of one or more genes selected from group (vi) or group (vii), or the corresponding expression product, the subject is predicted to have a decreased likelihood of response to treatment with the EGFR inhibitor.

If the RNA transcript is that of one or more genes located near EGFR on chromosome 7p11.2, the gene or genes may, for example, be selected from the group consisting of CALM1P2, CCT6A, CHCHD2, ECOP, FKBP9L, GBAS, LANCL2, MRPS17, PHKG1, PSPH, SEC61G, and SUMF2, and for every unit of increased expression, the subject is predicted to have an increased likelihood of response to treatment with the EGFR inhibitor. Preferred genes in this group includes ECOP, LANCL2, and GBAS.

If the RNA transcript is that of one or more genes located near ERBB2 on chromosome 17q21.1, the gene or genes may, for example, be selected from the group consisting of C17orf37, CRK7, GRB7, GSDML, NEUROD2, PERLD1, PNMT, PPP1R1B, STARD3, TCAP, ZNFN1A3, and ZPBP2, and for every unit of increased expression, the subject is predicted to have an increased likelihood of response to treatment with the EGFR inhibitor. In a particular embodiment, the gene is PERLD1 and/or C17orf37.

If the RNA transcript is that of one or more genes located near ERBB3 on chromosome 12q.13, the gene or genes may, for example, be selected from the group consisting of CDK2, FLJ14451, MBC2, MLC1SA, PS2G4, RAB5B, RPL41, RPS26, SILV, SUOX, and ZNFN1A4, and for every unit of increased expression, the subject is predicted to have an increased likelihood of response to treatment with the EGFR inhibitor. In a preferred embodiment, the cancer cells additionally express ERBB3. In a particular embodiment, the gene is RPS26 and/or PS2G4.

In the RNA transcript is that of one or more genes located near ERBB4 on chromosome 2q33.3-q34, the gene or genes may, for example, be selected from the group consisting of ACADL, CPS1, FLJ23861, LANCL1, MYL1, PF20, RPE, SNAI1L1, and ZNFN1A2, and for every unit of increased expression, the subject is predicted to have an increased likelihood of response to treatment with the EGFR inhibitor. In a preferred embodiment, the cancer cells additionally express ERBB4. In a particular embodiment, the gene is CPS1 and/or ZNFN1A2.

If the RNA transcript is that of one or more genes involved in ADCC and/or one or more gene markers of immune or inflammatory cells, the gene or genes may, for example, be selected from the group consisting of CD68, CD8A, CD8B1, CDH1, FCGR1A, FCGR1B, FCGR1C, FCGR2A, FCGR2B, FCGR3A, FCGR3B, GZMB, IFNG, IL12B, IL2, ITGAL, ITGB2, KLRK1, NCAM1, PTPRC, and TGFB1, and for every unit of increased expression, the subject is predicted to have an increased likelihood of response to treatment with the EGFR inhibitor. In a particular embodiment the gene is FCGR3A, ITGB2 or NCAM1.

If the RNA transcript is that of one or more genes associated with tumor cell invasion, the gene or genes may, for example, be selected from the group consisting of ANPEP, CMET, CTNND1, PTP4A3, PAI1, TIMP1, TIMP2, TIMP3, SLPI and PTTG1, and for every unit of increased expression, the subject is predicted to have a decreased likelihood of response to treatment with the EGFR inhibitor.

If the RNA transcript is that of one or more genes preferentially expressed in late stage tumors, the gene can, for example, be EPHB2 and/or GDF15, and for every unit of increased expression, the subject is predicted to have a decreased likelihood of response to treatment with the EGFR inhibitor.

For all aspects, the subject preferably is a human patient.

The cancer may, for example, be breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, melanoma, and brain cancer. Preferred types of cancer include breast cancer, non-small cell lung cancer (NSCLC), colorectal cancer, pancreatic cancer, prostate cancer, ovarian cancer, head and neck cancer, esophageal cancer, and glioblastoma multiforme, in particular head and neck squamous cell carcinoma (SCCHN).

EGFR inhibitors include, without limitation, Gefitinib, Erlotinib and Cetuximab.

In all embodiments, the biological sample includes tissue samples comprising cancer cells, such as fixed, paraffin-embedded, or fresh, or frozen tissues, which can be obtained from fine needle, core, or other types of biopsy, such as, for example, by fine needle aspiration, bronchial lavage, or transbronchial biopsy.

In another aspect, the invention concerns a method for preparing a personalized genomics profile for a human patient diagnosed with EGFR-expressing cancer comprising the steps of:
(a) determining in a biological sample containing cancer cells obtained from said patient the expression level of one or more RNA transcripts or their expression products, wherein the RNA transcript is of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, and

creating a report summarizing the information generated by step (a).

In a particular embodiment, the report includes a prediction of the likelihood that the patient will respond to treatment with an EGFR inhibitor.

In a preferred embodiment, the report includes a prediction of the likelihood that the patient will respond to treatment with Cetuximab.

In another embodiment, the report indicates that the patient has an increased likelihood of response to treatment with said EGFR inhibitor, if one or more genes selected from groups (i), (ii), (iii), (iv), and (v), or the corresponding expression products, show increased expression in said cancer cells.

In a further embodiment, the report indicates that said patient is predicted to have a decreased likelihood of response to treatment with said EGFR inhibitor, if one or more genes selected from group (vi) or group (vii), or the corresponding expression products, show increased expression in said cancer cells.

In a still further embodiment, the report includes a recommendation for a treatment modality of said patient.

In another embodiment, the report includes a recommendation to treat the patient with an EGFR inhibitor.

In yet another embodiment, the patient is treated with an EGFR inhibitor.

In a further aspect, the invention concerns an array comprising polynucleotides hybridizing to one or more genes generically or specifically listed throughout the specification. The polynucleotides include cDNAs and oligonucleotides, and may include more than one polynucleotide hybridizing to the same gene.

In a particular embodiment, at least one of the polynucleotides comprises an intron-based sequence the expression of which is correlates with the expression of a exon sequence of the same gene.

In a further aspect, the invention concerns a method of using a gene selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, to predict responsiveness of a patient diagnosed with EGFR-expressing cancer to treatment with an EGFR inhibitor, comprising predicting an increased likelihood of responsiveness if the expression level of one or more genes from groups (i)-(v) is elevated in the cancer, and predicting a decreased likelihood of responsiveness if the expression level of one or more genes from group (vi) or group (vii) is elevated in the cancer.

In a different aspect, the invention concerns a method of predicting the likelihood of responsiveness of a patient diagnosed with an EGFR-expressing cancer to treatment with an EGFR inhibitor, comprising
identifying evidence of elevated expression of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein
evidence of elevated expression of one or more genes from groups (i)-(v) indicates that the patient has an increased likelihood of responsiveness to treatment with said EGFR inhibitor, and
evidence of elevated expression of one or more genes from group (vi) or group (vii) indicates that the patient has a decreased likelihood of responsiveness to treatment with said EGFR inhibitor.

In a different aspect the invention concerns a report comprising a summary of the normalized expression levels of an RNA transcript or its expression products in a cancer cell obtained from a subject, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and evidence of elevated expression of one or more genes from group (vi) or group (vii) indicates that said subject has a decreased likelihood of response to treatment with said EGFR inhibitor.

In a different aspect the invention concerns a report comprising a prediction of the response of a subject diagnosed with EGFR positive cancer to treatment with an EGFR inhibitor based on a determination of the normalized expression levels of an RNA transcript or its expression products in a cancer cell obtained from said subject, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and evidence of elevated expression of one or more genes from group (vi) or group (vii) indicates that said subject has a decreased likelihood of response to treatment with said EGFR inhibitor.

In all aspects the report may be in electronic format.

In a different aspect the invention concerns a method of producing a report including gene expression information about a cancer cell obtained from a subject comprising the steps of: (a) determining normalized expression levels of an RNA transcript or its expression products in a cancer cell obtained from said patient, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and evidence of elevated expression of one or more genes from group (vi) or group (vii) indicates that said subject has a decreased likelihood of response to treatment with said EGFR inhibitor.; and (b) creating a report summarizing said information.

In a different aspect the invention concerns a kit comprising one or more of (1) extraction buffer/reagents and protocol; (2) reverse transcription buffer/reagents and protocol; and (3) qPCR buffer/reagents and protocol suitable for performing the methods of this invention. The kit may comprise data retrieval and analysis software.

### DETAILED DESCRIPTION OF THE INVENTION

Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); and Webster's New World™ Medical Dictionary, 2nd Edition, Wiley Publishing Inc., 2003, provide one skilled in the art with a general guide to many of the terms used in the present application. For purposes of the present invention, the following terms are defined below.

Gene symbols written in this application using all capital letters refer to human genes to which such symbol has been assigned as its Official Symbol by The Human Genome Organisation (HUGO) Gene Nomenclature Committee.

The term “invasion” means those biological processes that contribute to the ability of tumor cells to infiltrate into adjacent normal tissue and ultimately to metastasize to distant sites by transport through the circulatory and lymphatic systems.

The term “RT-PCR” has been variously used in the art to mean reverse-transcription PCR (which refers to the use of PCR to amplify mRNA by first converting mRNA to double stranded cDNA) or real-time PCR (which refers to ongoing monitoring in ‘real-time’ of the amount of PCR product in a reaction in order to quantify the amount of PCR target sequence initially present. As used herein, the term “RT-PCR” means reverse transcription PCR. The term “quantitative RT-PCR” (qRT-PCR) means real-time PCR applied to determine the amount of MRNA initially present in a sample.

The term “response” means any measure of patient response to treatment with a drug including those measures ordinarily used in the art, such as complete pathologic response, partial response, stable disease, time to disease progression, etc.

The term “microarray” refers to an ordered arrangement of hybridizable array elements, preferably polynucleotide probes, on a substrate. Microarrays include, without limitation, an ordered arrangement of polynucleotide probes on a microchip and a collection of polynucleotide coated beads on an arrangement of microfibers.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more unusual bases, such as inosine or one or more modified bases such as tritiated bases. Moreover the term includes DNAs (including cDNAs) and RNAs that contain one or more modified sugars, such as in locked nucleic acids. DNAs or RNAs with modified backbones, such as for example, phosphorothioates and peptide nucleic acids, and DNAs or RNAs with modified 5′ or 3′ phosphate moieties such as for example when conjugated with minor groove binders, are “polynucleotides” as that term is intended herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. Modified bases can be readily incorporated into chemically synthesized oligonucleotides made using automated synthesizers. Oligonucleotides can also be made by a variety of other methods, including in vitro recombinant DNA-mediated techniques and by expression of DNAs in cells and organisms.

The term “gene expression” describes the conversion of DNA gene sequence information into transcribed RNA (either the initial unspliced RNA transcript or the mature MRNA) or the encoded protein product. Gene expression can be monitored by measuring the levels of either RNA or protein products of the gene or subsequences.

The phrase “gene amplification” refers to a process by which multiple copies of a gene or gene fragment are formed in a particular cell or cell line. The duplicated region (a stretch of amplified DNA) is often referred to as “amplicon.” Often, the amount of the messenger RNA (mRNA) produced, i.e., the level of gene expression, also increases in proportion to the number of copies made of the particular gene expressed.

The term “prediction” is used herein to refer to the likelihood that a patient will respond to treatment, including with a drug or class of drugs and also to the nature and extent of those responses. The predictive methods of the present invention can be used clinically to make treatment decisions by choosing the most appropriate treatment modalities for any particular patient. The predictive methods of the present invention are valuable tools in predicting if a patient is likely to respond favorably to treatment with a drug in the EGFR inhibitor class.

The term “tumor,” as used herein, refers to all neoplastic cell growth and proliferation, whether malignant or benign, and all pre-cancerous and cancerous cells and tissues.

The terms “cancer” and “cancerous” refer to or describe the pathological condition in mammals that is typically characterized by rapid and unregulated cell growth. Examples of cancer include, but are not limited to, lung cancer, colorectal cancer, breast cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer. Preferred cancers are EGFR-expressing cancers, including, without limitation, non-small cell lung cancer (NSCLC), colorectal cancer, breast cancer, pancreatic cancer, prostate cancer, ovarian cancer, head and neck cancer (including head and neck squamous cell carcinoma, SCCHN), esophageal cancer, and glioblastoma multiforme

The “pathology” of cancer includes all phenomena that compromise the well-being of the patient. This includes, without limitation, abnormal or uncontrollable cell growth, invasion of surrounding normal tissues, metastasis, interference with the normal functioning of neighboring cells, release of cytokines or other secretory products at abnormal levels, suppression or aggravation of inflammatory or immunological response, neoplasia, premalignancy, malignancy, and invasion.

In the context of the present invention, reference to “one or more” genes is used herein to mean any one, two, three, four, five, etc. of the listed genes, in any combination.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in a mature RNA product (B. Lewin. Genes IV Cell Press, Cambridge Mass. 1990). In theory the term “intron” refers to any segment of DNA that is transcribed but is removed from within the initial transcript by splicing together the exons on either side of it. Operationally, the exon sequences of a gene occur in the MRNA sequence as defined by Ref. SEQ ID numbers. Operationally, intron sequences of a gene are sequences bracketed by exon sequences and having GT and AG splice consensus sequences at their 5′ and 3′ boundaries.

### DETAILED DESCRIPTION

### Genes and Gene Sets Predictive of Patient Response to EGFR Inhibitor Treatment

Upon binding one of its ligands, which include epidermal growth factor (EGF) and transforming growth factor alpha (TGF-α), the EGF receptor (EGFR) is activated via dimerization, either with another EGFR protein molecule or with another member of the EGFR receptor family, ERBB2, ERBB3 or ERBB4. Activated EGFR signals through a branched pathway that is regulated at multiple levels and influenced by activities in other cellular signaling pathways. Signaling through EGFR in turn affects multiple aspects of tumorigenesis including cell proliferation, angiogenesis and inhibition of apoptosis. Defects in this signaling network can result in overactive signaling which may cause tumorigenesis and cancer. The effectiveness of EGFR inhibition in cancer treatment may depend on the expression status of multiple genes in the signaling networks.

The present invention provides genes and gene sets useful in predicting the response of a subject diagnosed with cancer to treatment with an EGFR inhibitor. In particular, various sets of genes were assembled based on the involvement of their expression products in response to EGFR inhibitor drugs. Gene specific probe primer sets were designed based on gene exon and/or intron sequences. These probe primer sets may be used in conjunction with a variety of clinical samples to identify particular genes that in their expression predict the likelihood that a patient will show a beneficial response to an EGFR inhibitor drug.

In one aspect, genes located near EGFR (on chromosome 7p11.2) have been identified, the expression level of which correlates with the response of patients to treatment with an EGFR inhibitor. In particular, it has been found that a patient diagnosed with an EGFR-expressing tumor is more likely to respond to treatment with an EGFR inhibitor, if the tumor additionally expresses one or more of the following genes: CALM1P2, CCT6A, CHCHD2, ECOP, FKBP9L, GBAS, LANCL2, MRPS17, PHKG1, PSPH, SEC61G, and SUMF2. Preferably, an increased likelihood of patient response is predicted, if the patient's tumor expresses, in addition to EGFR, at least one, at least two, or at least three, or at least four, or at least five, or at least six, or at least seven, or at least eight, or at least nine, or at least ten, or all of the listed genes, in any combination. More preferably, an increased likelihood of patient response is predicted if the patient's tumor shows an increased level of expression of both of EGFR and ECOP, or EGFR and LANCL2, or EGFR and GBAS.

In another aspect, genes located near ERBB2 (chromosome 17q21.1) have been identified, the expression level of which correlates with the response of cancer patients to treatment with an EGFR inhibitor. In particular, a patient diagnosed with a tumor that expresses EGFR, is more likely to show a beneficial response to treatment with an EGFR inhibitor, if the tumor additionally expresses ERBB2 or one or more genes located near ERBB2 on chromosome 17, such as, for example, one or more of the following genes: C17orf37, CRK7, GRB7, GSDML, NEUROD2, PERLD1, PNMT, PPP1R1B, STARD3, TCAP, ZNFN1A3, and ZPBP2.

In yet another aspect, genes located near ERBB3 (chromosome 12q.13) have been identified, the expression level of which correlates with the response of cancer patients to treatment with an EGFR inhibitor. In particular a patient diagnosed with a tumor that expresses EGFR, is more likely to show a beneficial response to treatment with an EGFR inhibitor, if the tumor additionally expresses ERBB3 or one or more genes located near ERBB3 on chromosome 12, such as, for example, one or more of the following genes: CDK2, FLJ14451, MBC2, MLC1SA, PS2G4, RAB5B, RPL41, RPS26, SILV, SUOX, and ZNFN1A4.

In a further aspect, genes located near ERBB4 (chromosome 2q33.3-q34) have been identified, the expression level of which correlates with the response of cancer patients to treatment with an EGFR inhibitor. In particular, a patient diagnosed with a tumor that expresses EGFR, is more likely to show a beneficial response to treatment with an EGFR inhibitor, if the tumor additionally expresses ERBB4 or one or more genes located near ERBB4 on chromosome 2, such as, for example, one or more of the following genes: ACADL, CPS1, FLJ23861, LANCL1, MYL1, PF20, RPE, SNAI1L1, and ZNFN1A2.

The increased expression of one or more genes characteristic of immune or inflammatory cells or associated with ADCC in a sample from a patient's tumor indicates that the patient is more likely to have a beneficial response to treatment with an EGFR inhibitor than a patient whose tumor is not characterized by an increased expression of such gene or genes. Genes in this group, which can be used as indicators of a beneficial patient response either alone or in any combination, include CD68, CD8A, CD8B1, CDH1, FCGR1A, FCGR1B, FCGR1C, FCGR2A, FCGR2B, FCGR3A, FCGR3B, GZMB, IFNG, IL12B, IL2, ITGAL, ITGB2, KLRK1, NCAM1, PTPRC, and TGFB1.

In a further aspect, genes associated with invasion have been identified, the expression level of which in a patient's EGFR positive tumor is indicative whether the patient is likely to show a beneficial response to treatment with an EGFR inhibitor. In particular, if a gene associated with invasion, such as ANPEP, CMET, CTNND1, PTP4A3, PAI1, TIMP1, TIMP2, TIMP3, SLPI and PTTG1 shows increased expression in such tumor, the patient is less likely to respond to such treatment than in the absence of such increased expression.

In a further aspect, genes whose expression is characteristic of late stage tumors have been identified. Increased expression of one or more of such genes, such as, for example, EPHB2, and GDF 15 in a patient's EGFR positive tumor indicates that the patient is likely to show resistance to treatment with an EGFR inhibitor. In particular, if one or more genes characteristic of late stage tumors are found to show increased expression in the patient's tumor, the patient is less likely to respond to such treatment than in the absence of increased expression of such gene or genes.

In a related aspect, genes whose reduced expression relative to normal cells and/or early stage tumors is characteristic of late stage tumors have been identified. Decreased expression of one or more of such genes such as, for example, CDH1 in a patient's EGFR positive tumor indicates that the patient is likely to show resistance to treatment with an EGFR inhibitor. In particular, if one or more genes whose reduced expression is characteristic of late stage tumors are found to show decreased expression in the patient's tumor, the patient is less likely to respond to such treatment than in the absence of decreased expression of such gene or genes.

In a still further aspect, genes indicative of rate of cell proliferation have been identified. Changes in the expression level of genes that mark proliferating cells, such as, for example BUB1, in a patient's EGFR positive tumor are indicative of changes in the likelihood that said patient will respond to EGFR inhibitors.

The predictive genes identified or any gene group formed by particular combination of the predictive genes can be used alone, or can be used together with other predictive indicators. Other predictive indicators may include the expression of other genes and gene groups and may also include clinical variables including tumor size, stage and grade.

Alone or in combination with other prognostic indicators, the expression level of genes and gene groups of the present invention can be used to create an equation that yields a quantitative indicator of likelihood of response to EGFR inhibitors. This formula may differentially weight the expression levels of particular genes and may in addition take into account other variables such as clinical variables (e.g., number of involved lymph nodes and/or site(s) of metastasis).

EGFR Expressing Tumors and EGFR Inhibitors

There are a number of tumor types characterized by the expression of EGFR, including, without limitation, non-small cell lung cancer (NSCLC), colorectal cancer, breast cancer, pancreatic cancer, prostate cancer, ovarian cancer, head and neck cancer (including head and neck squamous cell carcinoma, SCCHN), esophageal cancer, and glioblastoma multiforme. See, for example, Ciardello and Tortola, Eur. J. Cancer 39:1348-1354 (2003) and Salomon et al., Crit. Rev. Oncol. Hematol. 19:183-232 (1996).

EGFR inhibitor drugs on the market include Gefitinib (IRESSA®, AstraZeneca), Erlotinib (TARCEVA®, Genentech, Inc.), and Cetuximab (ERBITUX®, ImClone Systems, Inc.). Phase I clinical trials have confirmed the responsiveness of colorectal cancer, breast cancer, SCCHN, glioblastoma multiforme, prostate cancer, ovarian cancer and renal cancer to treatment with at least one of these drugs. See, e.g., Ranson et al., J. Clin. Oncol. 20:2240-2250 (2002); Herbst et al., J. Clin. Oncol. 20:3815-3825 (2002); Baselga et al., J. Clin. Oncol. 20:4292-4302 (2002); Hidalgo et al., J. Clin. Oncol. 19:3267-3279 (2001); Prados et al., Proc. Am. Soc. Clin. Oncol. 22:99, Abstract 394 (2003).

Methods of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction” (Mullis et al., eds., 1994). The practice of the present invention will also employ, unless otherwise indicated, conventional techniques of statistical analyis such as the Cox Proportional Hazards model (see, e.g., Cox, D. R., and Oakes, D. (1984), Analysis of Survival Data, Chapman and Hall, London, N.Y.). Such techniques are explained fully in the literature.

In various embodiments of the invention, various technological approaches are available for determination of expression levels of the disclosed genes, including, without limitation, RT-PCR, microarrays, serial analysis of gene expression (SAGE) and Gene Expression Analysis by Massively Parallel Signature Sequencing (MPSS), which will be discussed in detail below. In particular embodiments, the expression level of each gene may be determined in relation to various features of the expression products of the gene including exons, introns, protein epitopes and protein activity. In other embodiments, the expression level of a gene may be inferred from measurement of copy number/amplification of a gene using techniques such as FISH or from analysis of the structure of the gene, for example from the analysis of the methylation pattern of gene's promoter(s). In addition, proteomic techniques can be readily used to based the analysis on determining the expression levels of the corresponding gene products. Such techniques, which are well known in the art, are specifically included within the scope herein.

### I. Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

### a. Reverse Transcriptase PCR (RT-PCR)

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g., formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan®) PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thernocycler. During amplification, laser-induced fluorescent signal is detected at the CCD. The system includes software for running the instrument and for analyzing the data.

5′-assay data are initially expressed as CT, or the threshold cycle. As discussed above, fluorescence values are recorded during every cycle and represent the amount of product amplified to that point in the amplification reaction. The point when the fluorescent signal is first recorded as statistically significant is the threshold cycle (C_{T}).

To minimize errors and the effect of sample-to-sample variation, RT-PCR is usually performed using one or more reference genes as internal standards. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPD) and β-actin (ACTB).

A more recent variation of the RT-PCR technique is real time quantitative RT-PCR (qRT-PCR), which measures PCR product accumulation through a dual-labeled fluorigenic probe (i.e., TaqMant probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g., Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles {for example: T. E. Godfrey et al. J. Molec. Diagnostics 2: 84-91 (2000); K. Specht et al., Am. J. Pathol. 158: 419-29 (2001); Cronin et al., Am J Pathol 164:35-42 (2004)}. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

### b. Microarrays

Differential gene expression can also be identified, or confirmed using microarray techniques. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g., formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization at each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology.

The development of microarray methods for large-scale analysis of gene expression makes it possible to search systematically for molecular markers of cancer classification and outcome prediction in a variety of tumor types.

### C. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g., Velculescu et al., Science 270:484-487 (1995); and Velculescu et al., Cell 88:243-51 (1997).

### d. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

### e. General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles (for example: T. E. Godfrey et al,. J. Molec. Diagnostics 2: 84-91 [2000]; K. Specht et al., Am. J. Pathol. 158: 419-29 [2001]). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the patient on the basis of the characteristic gene expression pattern identified in the tumor sample examined, dependent on the predicted likelihood of cancer recurrence.

### f. Reference Gene Set

An important aspect of the present invention is to use the measured expression of certain genes by breast cancer tissue to provide prognostic or predictive information. For this purpose it is necessary to correct for (normalize away) both differences in the amount of RNA assayed and variability in the quality of the RNA used. Well known housekeeping genes such as β-actin, GAPD, GUS, RPLO, and TFRC can be used as reference genes for normalization. Reference genes can also be chosen based on the relative invariability of their expression in the study samples and their lack of correlation with clinical outcome. Alternatively, normalization can be based on the mean or median signal (CT) of all of the assayed genes or a large subset thereof (global normalization approach). Below, unless noted otherwise, gene expression means normalized expression.

### g. Primer and Probe Design

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g., about 50 to 70° C. are typically preferred.

For further guidelines for PCR primer and probe design see, e.g., Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

### II. Sources of Biological Material

Treatment of cancer often involves resection of the tumor to the extent possible without severely compromising the biological function of the patient. As a result, tumor tissue is typically available for analysis following initial treatment of the tumor, and this resected tumor has most often been the sample used in expression analysis studies.

Expression analysis can also be carried out on tumor tissue obtained through other means such as core, fine needle, or other types of biopsy.

For particular tumor types, tumor tissue is appropriately obtained from biological fluids using methods such as fine needle aspiration, bronchial lavage, or transbronchial biopsy.

Particularly in relatively advanced tumors, circulating tumor cells (CTC) are sometimes found in the blood of cancer patients. CTC recovered from blood can also be used as a source of material for expression analysis.

Cellular constituents, including RNA and protein, derived from tumor cells have been found in biological fluids of cancer patients, including blood and urine. Circulating nucleic acids and proteins may result from tumor cell lysis and may be subjected to expression analysis.

These and all other sources of tumor or tumor cells are collectively referred to as “biological material,” or “biological sample.”

### III. Algorithms and Statistical Methods

When quantitative RT-PCR (qRT-PCR) is used to measure mRNA levels, mRNA amounts are expressed in C_{T} (threshold cycle) units (Held et al., Genome Research 6:986-994 (1996)). The averaged sum of C_{T}s for the reference mRNAs is arbitrarily set (e.g., to zero), and each measured test mRNA C_{T} is given relative to this fixed reference. For example, if, for a particular patient tumor specimen the average of CTS of the reference genes found to be 31 and C_{T} of test gene X is found to be 35, the reported value for gene X is −4 (i.e., 31-35).

The normalized data can be used to analyze correlation between the expression level of particular mRNAs and patient response. Standard statistical methods can be applied to identify those genes, for which the correlation between expression and a beneficial patient response, in a univariate analysis, is statistically significant. These genes are markers of outcome, given the existing clinical status. Multivariate analysis can be applied to identify sets of genes, the expression levels of which, when used in combination, are better markers of outcome (patient response) than the individual genes that constitute the sets.

Further, it is possible to define groups of genes known or suspected to be associated with particular aspects of the molecular pathology of cancer. A gene can be assigned to a particular group based either on its known or suspected role in a particular aspect of the molecular biology of cancer or based on its co-expression with another gene already assigned to a particular group. Co-pending U.S. Patent Application 60/561,035 defines several such groups and further shows that the definition of such groups (also termed axis or subset) is useful in that it supports particular methods of data analysis and the elaboration of mathematical algorithms, which in turn yields a more powerful predictors of outcome than can be formulated if these groups are not defined.

### IV. Clinical Application of Data

The methods of this invention can be performed as a self-contained test. Individual markers of the invention identified by univariate analysis or sets of markers of the invention (e.g., identified by multivariate analysis) are useful predictors of clinical outcome. Alternatively the markers can be applied as predictive elements of a test that could include other predictive indicators including a) other genes and/or gene groups, or b) other clinical indicators such as tumor stage and grade. Other genes or gene groups that can be beneficially combined with the predictive genes and gene sets of the present invention are included, for example, in United States Patent Application Publication Nos. 20040157255, published Aug. 12, 2004, and 20050019785, published Jan. 27, 2005, the entire disclosures of which are hereby expressly incorporated by reference.

### V. Kits of the Invention

The methods of this invention, when practiced for commercial diagnostic purposes are typically performed in a CLIA-approved clinical diagnostic laboratory. The materials for use in the methods of the present invention are suited for preparation of kits produced in accordance with well known procedures. The invention thus provides kits or components thereof, such kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic or predictive information are also properly potential components of kits.

### VI. Reports of the Invention

The methods of this invention, when practiced for commercial diagnostic purposes generally produce a report or summary of the normalized expression levels of one or more of the selected genes. The methods of this invention will produce a report containing a estimation of the likelihood of response of a subject diagnosed with an EGFR cancer to treatment with an EGFR inhibitor based on a determination of the level of expression of one or more selected genes. The method and report can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer.

The methods provided by the present invention may also be automated in whole or in part.

All aspects of the present invention may also be practiced such that a limited number of additional genes that are co-expressed with the disclosed genes, for example as evidenced by high Pearson correlation coefficients, are included in a prognostic or predictive test in addition to and/or in place of disclosed genes.

1. A method for predicting the response of a subject diagnosed with EGFR positive cancer to treatment with an EGFR inhibitor, comprising determining the expression level of one or more RNA transcripts or their expression products in a biological sample containing cancer cells obtained from said subject, wherein the RNA transcript is of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, wherein
(a) for every unit of increased expression of one or more genes selected from groups (i), (ii), (iii), (iv), and (v), or the corresponding expression product, said subject is predicted to have an increased likelihood of response to treatment with said EGFR inhibitor; and
(b) for every unit of increased expression of one or more genes selected from group (vi) or group (vii), or the corresponding expression product, said subject is predicted to have a decreased likelihood of response to treatment with said EGFR inhibitor.

2. The method of claim 1 wherein said RNA transcript is that of one or more genes located near EGFR on chromosome 7p11.2.

3. The method of claim 2 wherein said gene is selected from the group consisting of CALM1P2, CCT6A, CHCHD2, ECOP, FKBP9L, GBAS, LANCL2, MRPS17, PHKG1, PSPH, SEC61G, and SUMF2, and for every increment of increased expression, said subject is predicted to have an increased likelihood of response to treatment with said EGFR inhibitor.

4. The method of claim 3 wherein said gene is ECOP, LANCL2, or GBAS, or any combination thereof.

5. The method of claim 1 wherein said RNA transcript is that of ERBB2 or one or more genes located near ERBB2 on chromosome 17q21.1.

6. The method of claim 5 wherein said gene is selected from the group consisting of C17orf37, CRK7, GRB7, GSDML, NEUROD2, PERLD1, PNMT, PPP1R1B, STARD3, TCAP, ZNFN1A3, and ZPBP2, and for every unit of increased expression, said subject is predicted to have an increased likelihood of response to treatment with said EGFR inhibitor.

7. The method of claim 6 wherein said gene is PERLD1 and/or C17orf37.

8. The method of claim 6 wherein said cancer cells additionally express ERBB2.

9. The method of claim 1 wherein said RNA transcript is that of one or more genes located near ERBB3 on chromosome 12q.13.

10. The method of claim 9 wherein said gene is selected from the group consisting of CDK2, FLJ14451, MBC2, MLC1SA, PA2G4, RAB5B, RPL41, RPS26, SILV, SUOX, and ZNFN1A4, and for every unit of increased expression, said subject is predicted to have an increased likelihood of response to treatment with said EGFR inhibitor.

11. The method of claim 10 wherein said gene is RPS26 and/or PS2G4.

12. The method of claim 11 wherein said cancer cells additionally express ERBB3.

13. The method of claim 1 wherein said RNA transcript is that of one or more genes located near ERBB4 on chromosome 2q33.3-q34.

14. The method of claim 12 wherein said gene is selected from the group consisting of ACADL, CPS1, FLJ23861, LANCL1, MYL1, PF20, RPE, SNAI1L1, and ZNFN1A2, and for every unit of increased expression, said subject is predicted to have an increased likelihood of response to treatment with said EGFR inhibitor.

15. The method of claim 6 wherein said gene is CPS1 and/or ZNFN1A2.

16. The method of claim 14 wherein said cancer cells additionally express ERBB4.

17. The method of claim 1 wherein said RNA transcript is that of one or more genes involved in ADCC and/or one or more or gene markers of immune or inflammatory cells.

18. The method of claim 17 wherein said gene is selected from the group consisting of CD68, CD8A, CD8B1, CDH1, FCGR1A, FCGR1B, FCGR1C, FCGR2A, FCGR2B, FCGR3A, FCGR3B, GZMB, IFNG, IL12B, IL2, ITGAL, ITGB2, KLRK1, NCAM1, PTPRC, and TGFB1, and for every unit of increased expression, said subject is predicted to have an increased likelihood of response to treatment with said EGFR inhibitor.

19. The method of claim 18 wherein said gene is FCGR3A, ITGB2, and NCAM1.

20. The method of claim 1 wherein said RNA transcript is that of one or more genes associated with tumor cell invasion.

21. The method of claim 20 wherein said gene is selected from the group consisting of ANPEP, CMET, CTNND1, PTP4A3, PAI1, TIMP1, TIMP2, TIMP3, SLPI and PTTG1, and for every unit of increased expression, said subject is predicted to have a decreased likelihood of response to treatment with said EGFR inhibitor.

22. The method of claim 1 wherein said RNA transcript is that of one or more genes preferentially expressed in late stage tumors.

23. The method of claim 22 wherein said gene is selected from the group consisting of EPHB2 and GDF15, and for every unit of increased expression, said subject is predicted to have a decreased likelihood of response to treatment with said EGFR inhibitor.

24. The method of claim 1 wherein said subject is a human patient.

25. The method of claim 24 wherein said cancer is selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer.

26. The method of claim 25 wherein said cancer is selected from the group consisting of breast cancer, non-small cell lung cancer (NSCLC), colorectal cancer, pancreatic cancer, prostate cancer, ovarian cancer, head and neck cancer, esophageal cancer, and glioblastoma multiforme.

27. The method of claim 26 wherein said head and neck cancer is head and neck squamous cell carcinoma (SCCHN).

28. The method of claim 1 wherein said EGFR inhibitor is selected from the group consisting of Gefitinib, Erlotinib and Cetuximab.

29. The method of claim 1 wherein said biological sample is a tissue sample comprising cancer cells.

30. The method of claim 29 wherein said tissue is fixed, paraffin-embedded, or fresh, or frozen.

31. The method of claim 30 where the tissue is from fine needle, core, or other types of biopsy.

32. The method of claim 30 wherein the tissue sample is obtained by fine needle aspiration, bronchial lavage, or transbronchial biopsy.

33. The method of claim 30 wherein said tissue is a fixed, paraffin-embedded tissue sample.

34. The method of claim 1 wherein the expression level of said RNA transcript or transcripts is determined by RT-PCR.

35. The method of claim 1 wherein the expression level of said expression product or products is determined by immunohistochemistry.

36. The method of claim 1 wherein the expression level of said expression product or products is determined by proteomics techniques.

37. The method of claim 1 wherein the assay for the measurement of said RNA transcripts or their expression products is provided in the form of a kit or kits.

38. A method for preparing a personalized genomics profile for a human patient diagnosed with EGFR-expressing cancer comprising the steps of:
(a) determining in a biological sample containing cancer cells obtained from said patient the expression level of one or more RNA transcripts or their expression products in a biological sample containing cancer cells obtained from said subject, wherein the RNA transcript is of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, and (b) creating a report summarizing the information generated by step (a).

39. The method of claim 38 wherein said report includes a prediction of the likelihood that said patient responds to treatment with an EGFR inhibitor.

40. The method of claim 39 wherein said report indicates that said patient has an increased likelihood of response to treatment with said EGFR inhibitor, if one or more genes selected from groups (i), (ii), (iii), (iv), and (v), or the corresponding expression products, show increased expression in said cancer cells.

41. The method of claim 39 wherein said report indicates that said patient has a decreased likelihood of response to treatment with said EGFR inhibitor, if one or more genes selected from group (vi) or group (vii), or the corresponding expression products, show increased expression in said cancer cells.

42. The method of claim 39 wherein said cancer cells are obtained from a solid tumor.

43. The method of claim 42 wherein said tumor is selected from the group consisting of breast cancer, lung cancer, colorectal cancer, pancreatic cancer, prostate cancer, ovarian cancer, head and neck cancer, esophageal cancer, glioblastoma multiforme, hepatocellular cancer, gastric cancer, cervical cancer, liver cancer, bladder cancer, cancer of the urinary tract, thyroid cancer, renal cancer, carcinoma, melanoma, and brain cancer.

44. The method of claim 42 wherein said cancer cells are obtained from a fixed, paraffin-embedded biopsy sample of said tumor.

45. The method of claim 39 wherein said report includes a recommendation for a treatment modality of said patient.

46. The method of claim 45 wherein said report includes a recommendation to treat said patient with an EGFR inhibitor.

47. The method of claim 46 further comprising the step of treating said patient with an EGFR inhibitor.

48. An array comprising polynucleotides hybridizing to one or more genes according to claim 1, immobilized on a solid surface.

49. The array of claim 48 comprising polynucleotides hybridizing to any of the group of genes.

50. The array of claim 48 or claim 49 wherein said polynucleotides are cDNAs.

51. The array of claim 48 or claim 49 wherein said polynucleotides are oligonucleotides.

52. The array of claim 48 or claim 49 comprising more than one polynucleotide hybridizing to the same gene.

53. The array of claim 48 or claim 49 wherein at least one of said polynucleotides comprises an intron-based sequence the expression of which is correlates with the expression of a corresponding exon sequence.

54. A method of using a gene selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, to predict responsiveness of a patient diagnosed with EGFR-expressing cancer to treatment with an EGFR inhibitor, comprising predicting an increased likelihood of responsiveness if the expression level of one or more genes from groups (i)-(v) is elevated in said cancer, and predicting a decreased likelihood of responsiveness if the expression level of one or more genes from group (vi) or group (vii) is elevated in said cancer.

55. A method of predicting the likelihood of responsiveness of a subject diagnosed with an EGFR-expressing cancer to treatment with an EGFR inhibitor, comprising
identifying evidence of elevated expression of one or more genes selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein
evidence of elevated expression of one or more genes from groups (i)-(v) indicates that said subject has an increased likelihood of response to treatment with said EGFR inhibitor, and
evidence of elevated expression of one or more genes from group (vi) or group (vii) indicates that said subject has a decreased likelihood of response to treatment with said EGFR inhibitor.

56. A report comprising a summary of the normalized expression levels of an RNA transcript or its expression products in a cancer cell obtained from a subject, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and
evidence of elevated expression of one or more genes from group (vi) or group (vii) indicates that said subject has a decreased likelihood of response to treatment with said EGFR inhibitor.

57. A report comprising a prediction of the response of a subject diagnosed with EGFR positive cancer to treatment with an EGFR inhibitor based on a determination of the normalized expression levels of an RNA transcript or its expression products in a cancer cell obtained from said subject, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and
evidence of elevated expression of one or more genes from group (vi) or group (vii) indicates that said subject has a decreased likelihood of response to treatment with said EGFR inhibitor.

58. The report of claim 57 wherein said report is in electronic form.

59. A method of producing a report including gene expression information about a cancer cell obtained from a subject comprising the steps of:
(a) determining normalized expression levels of an RNA transcript or its expression products in a cancer cell obtained from said subject, wherein said RNA transcript is the RNA transcript of a gene or gene set selected from the group consisting of (i) genes located near EGFR on chromosome 7p11.2, (ii) ERBB2 and genes located near ERBB2 on chromosome 12q.13, (iii) ERBB3 and genes located near ERBB3 on chromosome 17q21.1; (iv) ERBB4 and genes located near ERBB4 on chromosome 7p11.2; (v) genes involved in ADCC and gene markers of immune or inflammatory cells; (vi) genes associated with tumor cell invasion; and (vii) genes characteristic of late stage tumors, or a corresponding gene product, wherein evidence of elevated expression of one or more genes from groups (i)-(v) indicates that said subject has an increased likelihood of response to treatment with said EGFR inhibitor, and
evidence of elevated expression of one or more genes from group (vi) or group (vii) indicates that said subject has a decreased likelihood of response to treatment with said EGFR inhibitor.; and
(b) creating a report summarizing said information.

60. A kit comprising one or more of (1) extraction buffer/reagents and protocol; (2) reverse transcription buffer/reagents and protocol; and (3) qPCR buffer/reagents and protocol suitable for performing the method of claim 1.

61. The kit of claim 60 further comprising data retrieval and analysis software.
